(19) United States
(12) Patent Application Publication
(10) Pub. No.: US 2023/0330021 A1
(43) Pub. Date: Oct. 19, 2023

(54) DRUG-LOADED POLYMER VESICLE HAVING ASYMMETRIC MEMBRANE STRUCTURE, PREPARATION METHOD THEREFOR, AND APPLICATION THEREOF IN PREPARATION OF DRUGS FOR TREATING ACUTE MYELOID LEUKEMIA

(71) Applicant: SOOCHOW UNIVERSITY, Suzhou (CN)

(72) Inventors: Zhiyuan ZHONG, Suzhou (CN); Fenghua MENG, Suzhou (CN)

(21) Appl. No.: 18/012,611

(22) PCT Filed: Feb. 1, 2021
(86) PCT No.: PCT/CN2021/074734

§ 371 Date: Jul. 7, 2023

(30) Foreign Application Priority Data
Sep. 14, 2020 (CN) 202010963969.4

(51) Int. Cl. A61K 31/704; A61K 9/127; A61P 35/02; A61K 31/475; A61K 31/136

(52) U.S. Cl. CPC A61K 9/1273; A61K 31/136; A61K 31/475; A61K 31/704; A61P 35/02; B82Y 5/00

(57) ABSTRACT

A drug-loaded polymer vesicle having an asymmetric membrane structure, a preparation method therefor, and an application thereof in preparation of drugs for treating acute myeloid leukemia. An amphiphilic triblock polymer having polyaspartic acid (PAsp), a targeted amphiphilic block polymer, and a small-molecule drug are assembled together to prepare the targeted small-molecule drug-loaded polymer vesicle having the asymmetric membrane structure. The drug-loaded polymer vesicle has many unique advantages, comprising small size, simple and controllable preparation, reversible crosslinking, in-vivo stability, targeted delivery, high enrichment concentration of intracellular drugs, reduction sensitivity, efficient killing of tumor cells, remarkable tumor growth inhibition effect, etc., and has an effective inhibition effect on both acute myelogenous leukemia cell strains and patient cells. Therefore, the polymer vesicle is expected to become a simple and multifunctional nanoplatform for efficient and specific targeted delivery of the drugs to the tumor cells.

### TECHNICAL FIELD

The invention belongs to the technical field of polymer nano-drugs, and in particular relates to a reversible loading the small molecule drug on the vesicle formed by a cross-linked biodegradable polymer, and a preparation method therefor, and use thereof.

### BACKGROUND TECHNIQUE

Acute myeloid leukemia is a common blood disease, accounting for about ⅓ of all leukemias, and its typical feature is the abnormal proliferation and differentiation of myeloid cells. If not detected and treated in time, acute myeloid leukemia can induce severe symptoms of acute bone marrow failure, leading to death within weeks or months. In 2018, there were 437,033 new cases globally and 309,006 deaths. Therefore, the treatment of leukemia is facing a very serious situation. In the past 30 years, the standard treatment regimen for acute myeloid leukemia has not developed by leaps and bounds. It has always been a combination treatment regimen of cytarabine and daunorubicin (7+3), and the treatment effect is also unsatisfactory, with a 5-year survival rate of about 27%, and the recurrence rate exceeds 60%. Therefore, it becomes extremely urgent to seek new treatment options. With the continuous development of nano-drugs, nano-drugs have certain advantages in improving the efficacy of small-molecule drugs, especially in drug targeting, pharmacokinetics, route of administration, drug resistance, and side effects. Therefore, the key to the treatment of acute myeloid leukemia is to prepare nanocarriers with controllable physicochemical properties, stably load drugs, and increase the concentration of drugs in tumor cells through rational design.

### SUMMARY OF THE INVENTION

### Technical Problem

The purpose of the present invention is to disclose the amphiphilic triblock polymer and loading a drug on the vesicle formed by polymer, and a preparation method for, and use of. in particular relates to a reversible loading the small molecule drug on the vesicle formed by a cross-linked biodegradable polymer, and a preparation method for, and use of.

### Technical Solution

In order to achieve the above purpose, the present invention adopts the following technical solution:

The peptide targeting loading a drug on the vesicle formed by polymer with an asymmetric membrane structure, is obtained by amphiphilic triblock polymer and peptide targeting amphiphilic block polymer co-loading with small molecule drugs.

Application of drug-loaded vesicle formed by polymer with asymmetric membrane structure anti-acute myeloid leukemia drugs; drug-loaded vesicle formed by polymer with asymmetric membrane structure are prepared by loading small molecule drugs with amphiphilic triblock polymers.

Use of loading a drug on the vesicle formed by polymer with an asymmetric membrane structure in preparing anti-acute myeloid leukemia drugs; the loading a drug on the vesicle formed by polymer with an asymmetric membrane structure is obtained by amphiphilic triblock polymer loading small molecule drugs with amphiphilic block polymer.

In the present invention, the chemical structural formula of amphiphilic triblock polymer is as follows:

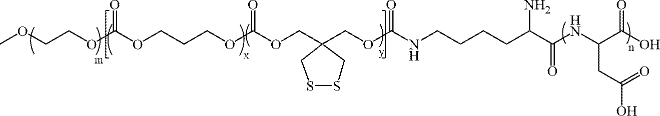

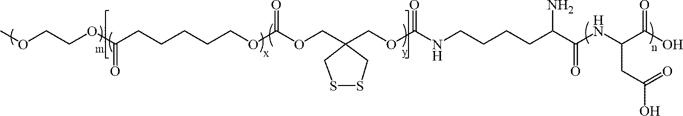

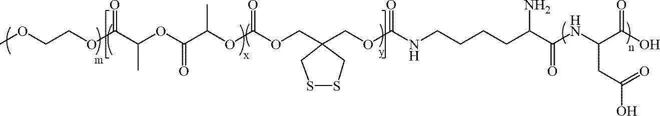

wherein, n is 5-20.

In the present invention, in the amphiphilic triblock polymer, the molecular weight of the hydrophilic chain segment is 3000-8000 Da; the molecular weight of the hydrophobic chain segment is 2.5-6 times that of the hydrophilic chain segment; the hydrophilic chain segment is polycarbonate (PTMC), polylactic acid (PLA) or polycaprolactone (PCL); the molecular weight of PDTC chain segment is 11%-30% of that of the hydrophobic chain segment. The molecular weight of PAsp is 17%-50% of that of PEG. The amphiphilic triblock polymer of the present invention has a hydrophilic chain segment (m chain segment), a hydrophobic chain segment (x+y chain segment), and a PAsp chain segment (n chain segment). The hydrophobic chain segment and the PAsp chain segment pass through group connection; the amphiphilic triblock polymers are denoted as PEG-P(TMC-DTC)-PAsp, PEG-P(CL-DTC)-PAsp, PEG-P(LA-DTC)-PAsp, and corresponding to the structural unit.

In the present invention, the amphiphilic triblock polymer is prepared from an amphiphilic block polymer. The preparation method includes the following steps: the terminal hydroxyl group of the amphiphilic block polymer is activated by p-nitrophenyl chloroformate, and then the amphiphilic triblock polymer is prepared with PAsp. In the amphiphilic block polymer, the molecular weight of PEG is 3000-8000 Da; the total molecular weight of the hydrophobic chain segment is 2.5-6 times of that of PEG; the total molecular weight of PDTC is 11%-30% of that of the hydrophobic chain segment.

The chemical structural formula of amphiphilic block polymer is as follows:

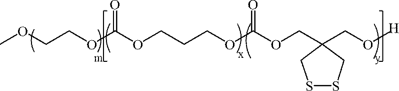

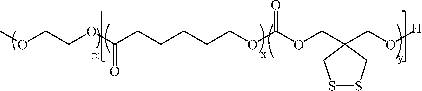

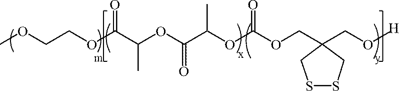

The chemical structural formula of PAsp is:

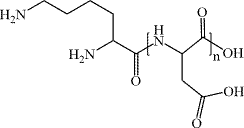

wherein, n is 5-15.

In the present invention, the targeting amphiphilic block polymer is obtained by Mal functional group or an NHS functional group to a targeting peptide; the specific method is a conventional method.

In the present invention, the targeting peptides include A6, CLL1 and iNGR. The sequence of A6 is KPSSPPEE, the sequence of CLL1 is CDLRSAAVC(C-C bridge), the sequence of iNGR is CRNGRGPDC(C-C bridge). The targeting peptide of the present invention is preferably A6.

In the present invention, the loading a drug on the vesicle formed by polymer is obtained by amphiphilic triblock polymer, and has an asymmetric membrane structure, the outer shell is a hydrophilic chain segment PEG, and the membrane layer is a reversibly cross-linked hydrophobic chain segment. The inner shell is PAsp, which can realize the efficient loading of positively charged small molecule drugs.

The loading a drug on the vesicle formed by polymer of the present invention, is obtained by drug and amphiphilic triblock polymer; or is obtained by drug, amphiphilic triblock polymer and the targeting amphiphilic block polymer. Specifically, the drug and the above-mentioned amphiphilic triblock polymer are used as raw materials to prepare non-targeted drug-loaded vesicle formed by polymer by a solvent replacement method. There are two preparation methods for. There are two preparation methods for the loading a drug on the vesicle formed by polymer: 1. pre-modification: preparing the targeting loading drug on the vesicle formed by polymer by a solvent displacement method using the drug, the amphiphilic triblock polymer and targeting amphiphilic block polymer as raw materials; 2. Post-modification: preparing the loading drug on the vesicle formed by polymer by a solvent displacement method using the drug, the amphiphilic triblock polymer and the Mal functional group or an NHS functional group amphiphilic block polymer as raw materials, then preparing the targeting loading drug on the vesicle formed by modified and targeted on the surface of the functionalized.

The preferred method for preparing targeted drug-loaded vesicle formed by polymer of the present invention is pre-modification. The amount of the targeted amphiphilic block polymer is 5%-35% of that of the molar sum of the amphiphilic triblock polymer and targeting amphiphilic block polymer; the amount of the functionalized amphiphilic block polymer is 5%-35% of that of the molar sum of the amphiphilic triblock polymer and targeting amphiphilic block polymer.

In the present invention, the small molecule drugs include vincristine sulfate (VCR), daunorubicin(DNR) or mitoxantrone(MTO). The small molecule drug of the present invention is preferably a VCR.

The present invention discloses a targeting/ non- targeting loading a drug on the vesicle formed by polymer with an asymmetric membrane structure, the non- targeting loading a drug on the vesicle formed by polymer is obtained by amphiphilic triblock polymer, the targeting loading a drug on the vesicle formed by polymer is obtained by amphiphilic triblock polymer/ the targeting amphiphilic block polymer; the above-mentioned loading a drug on the vesicle formed by polymer with an asymmetric membrane structure in preparing anti-acute myeloid leukemia drugs.

Loading a drug on the vesicle formed by polymer of the present invention is composed of drug and the vesicle formed by polymer, the vesicle formed by polymer is obtained by cross-linking of polymer; taking polycarbonate, A6, Mal functional group, and VCR as examples, the preparation method for the loading a drug on the vesicle formed by polymer of the present invention can be as follows:

The terminal hydroxyl group of PEG-P(TMC-DTC) is activated by p-nitrophenyl chloroformate, and then reacted with PAsp to obtain PEG-P(TMC-DTC)-PAsp.

Mal functional group is introduced into the PEG end of PEG-P(TMC-DTC) to obtain functionalized amphiphilic block polymer Mal-PEG-P(TMC-DTC); segmented polymer A6-PEG-P (TMC-DTC).

Using VCR, PEG-P(TMC-DTC)-PAsp as raw materials, reversible cross-linking and degradable non-targeting VCR-loaded polymer vesicle is prepared by solvent replacement method; or VCR, PEG-P(TMC-DTC)- PAsp and A6-PEG-P(TMC-DTC) are used as raw materials to prepare targeting VCR-loaded polymersomes by solvent displacement method.

The solution of PEG-P(TMC-DTC)-PAsp polymer can be injected into the VCR aqueous solution, and dialyzed after stirring to obtain reversibly cross-linked and degradable non-targeted VCR-loaded vesicle formed by polymer (cPS-VCR); In order to dissolve VCR in HEPES buffer (pH 6.8, 10 mM), then inject the DMSO solution of PEG-P(TMC-DTC)-PAsp polymer into it, stir evenly, and incubating at 37° C. cPS-VCR is obtained by dialysis against HEPES (pH 7.4, 10 mM).

The mixed solution of PEG-P(TMC-DTC)-PAsp and A6-PEG-P(TMC-DTC) polymer can be injected into the VCR aqueous solution, stirring and dialyzed to obtain a reversibly cross-linked and degradable target-loaded VCR. Polymersomes (A6-cPS-VCR); specifically, VCR was dissolved in HEPES buffer (pH 6.8, 10 mM), and then PEG-P(TMC-DTC)-PAsp and A6-PEG-P ( The mixed solution of TMC-DTC) polymer in DMSO solution is left to incubate at 37° C. after stirring evenly. A6-cPS-VCR is obtained by dialysis against HEPES (pH 7.4, 10 mM).

The vesicle formed by polymer in the present invention is reduction-sensitive reversible cross-linking, intracellular reversible cross-linking and biodegradable vesicle formed by polymer with negatively charged inner shell; the amphiphilic triblock polymer is PEG- Take P(TMC-DTC)-PAsp as an example, in which the TMC and DTC in the middle block are randomly arranged; PAsp has good biocompatibility, and the molecular weight of the PAsp segment is much smaller than that of PEG, which is obtained after self-assembly and cross-linking. The inner shell is a polymer vesicle with an asymmetric membrane structure of PAsp segments. The inner shell of the polymersome, PAsp, is negatively charged and can be used to compound positively charged small-molecule drugs. The vesicle membrane is a biodegradable and compatible PTMC, and the dithiopentane structure of the side chain is similar to the human body’s natural antioxidant lipoic acid, which can spontaneously form a reduction-sensitive reversible cross-linking, which not only ensures the drug in the blood. Stable and long-term circulation, it can also achieve rapid intracellular de-crosslinking and rapid release of drugs into target cells.

The invention encapsulates the small molecule medicine by electrostatic force, and can realize the efficient and stable encapsulation of the small molecule medicine. At the same time, the vesicle membrane cross-linked by disulfide is separated from the outside world, which can avoid the loss and toxic side effects caused by leakage and cell adhesion during the delivery process, and can be efficiently delivered to the lesion site, and the reducing agent glutathione ( Under the action of GSH), small molecule drugs are rapidly released to effectively kill tumor cells.

Small molecule drugs usually refer to chemical drugs with molecular weight < 1000 Da, which are relatively simple in structure and synthesis, stable in physical and chemical properties, usually non-immunogenic, and have low development cost and production difficulty. According to statistics, among the commonly used drugs, the number of small molecule drugs accounts for more than 98% of the total, and the market share of small molecule drugs is as high as 70%. However, it has inherent defects, such as usually distributed in various organs in the body, non-targeting and high side effects. The vesicles with asymmetric membrane structure in the present invention can overcome the above-mentioned defects and realize the efficient and specific targeted delivery of small molecule drugs. Protein drugs (or polypeptide drugs) generally act on the target on the cell surface, and the inhibitory protein has strong specificity, but it is generally difficult to enter the cell and complement each other with small molecule drugs. The vesicles with asymmetric membrane structure in the present invention, the inner shell is PAsp, can not only load small molecule drugs, but also protein drugs (or polypeptide drugs). However, protein drugs (or polypeptide drugs) have various types, complex molecular structures, and special requirements for biological activity and immunogenicity evaluation, which make the production process and quality control of protein-loaded drugs (or polypeptide drugs) vesicle formed by polymer with strong “complexity” and “specialty.” The small molecule drug-carrying vesicle formed by polymer in the present invention have many unique advantages, including simple and controllable preparation, in vivo stability, targeted delivery, low side effects, and significant tumor growth inhibitory effect, etc., and have clinical transformation prospects.

The invention discloses the application of the above-mentioned small molecule drug-loaded tumor-targeted, reversibly cross-linked and degradable vesicle formed by polymer in anti-tumor targeted therapy. Preferably, the tumor is acute myeloid leukemia AML.

### Advantageous Effects of the Invention

The present invention has the following advantages compared to the prior art:

1. The present invention designs a new vesicle formed by polymer for efficient loading of small molecule drugs and tumor-targeted delivery; firstly, an amphiphilic triblock polymer is synthesized and targeted amphiphilic block polymers, the polymer vesicle membranes are reversibly cross-linked biodegradable and biocompatible PTMC, PCL or PLA, and the side chain dithiopentane can provide reduction-sensitive reversible Cross-linking can not only ensure the long-term circulation of the drug in the blood, but also rapidly de-cross-link in the cells, releasing the drug into the target cells.

2. The inner shell of the polymer vesicle of the present invention is PAsp. Since the molecular weight of PAsp is smaller than that of the hydrophilic section of PEG, an asymmetric membrane structure in which the inner shell is PAsp is obtained after the polymer self-assembly and self-crosslinking, and the PAsp of the inner shell can be used for efficient loading of small molecule drugs.

3. The polymer vesicle shell of the present invention is PEG, which has targeting and can specifically bind to tumor cells; the small size of the polymer vesicle and the tumor-specific targeting enable the polymer vesicle to efficiently deliver small molecule drugs to tumor cells Inside.

4. The polymer vesicle carrier of the invention avoids the defects of the existing nanocarriers, such as poor circulation stability in vivo, low tumor cell selectivity, and low concentration of intracellular small molecule drugs.

5. The vesicle formed by polymer of the present invention have many unique advantages, including small size, simple and controllable preparation, excellent biocompatibility, high circulation stability in vivo, strong tumor cell specific selectivity, rapid drug release rate in cells and rapid killing Significant inhibitory effect on tumor cells and tumor growth, etc. Therefore, this vesicle system is expected to become a simple and all-in-one nanoplatform for efficient and specific targeted delivery of small molecule drugs to tumor cells.

### BRIEF DESCRIPTION OF THE DRAWINGS

FIG. 1 is the nuclear magnetic spectrum of PEG-P(TMC-DTC)-NPC in Example 1.

FIG. 2 is the nuclear magnetic spectrum of PEG-P(TMC-DTC)-PAsp in Example 1.

FIG. 3 is the nuclear magnetic spectrum of Mal-PEG-P(TMC-DTC) in Example 2.

FIG. 4 is the nuclear magnetic spectrum of A6-PEG-P(TMC-DTC) in Example 2.

FIG. 5A is the hydrodynamic particle size diagram and cryo-EM diagram of 4 kinds of A6-cPS-VCR in Example 4_{∘}

FIG. 5B shows the VCR release behavior of A6-cPS-VCR under reducing and nonreducing conditions in Example 5.

FIG. 5C shows the inhibition of cell proliferation of A6-cPS-VCR with different A6 surface densities in the acute myeloid leukemia cell line MV4-11 in Example 6.

FIG. 5D shows the endocytosis of Cy5-A6-cPS in the acute myeloid leukemia cell line MV4-11 in Example 7.

FIG. 6 shows the proliferation inhibition of A6-cPS-VCR, cPS-VCR and VCR in CD44-positive acute myeloid leukemia cell lines MV4-11, HL-60 and SHI-1 in Example 8.

FIG. 7 shows the proliferation inhibition of A6-cPS-VCR, cPS-VCR and VCR in CD44-negative acute myeloid leukemia cell lines YNH-1 and OCI-AML-3 in Example 8.

FIG. 8 shows the proliferation inhibition of MV4-11 by A6-cPS-DNR, cPS-DNR and DNR in Example 8.

FIG. 9 shows the apoptosis of MV4-11 cells by A6-cPS-VCR, cPS-VCR and VCR in Example 8.

FIG. 10 shows the effects of A6-cPS-VCR, cPS-VCR and VCR on the cycle of MV4-11 cells in Example 8.

FIG. 11 shows the construction of the orthotopic MV4-11-GFP-Luc acute myeloid leukemia transplantation model mice in Example 9.

FIG. 12 shows the distribution of tumors by bioluminescence fluorescence imaging on the 10th day after modeling of the in situ MV4-11-GFP-Luc acute myeloid leukemia mice in Example 9.

FIG. 13 shows for 8 hours later Cy5 fluorescence imaging of femur, tibia, and ilium by injecting Cy5-A6-cPS and Cy5-cPS the tenth day after the model of the in situ MV4-11-GFP-Luc acute myeloid leukemia mice in Example 10.

FIG. 14 is the treatment workflow of the orthotopic MV4-11-GFP-Luc acute myeloid leukemia mouse transplantation model in the Netherlands in Example 11.

FIG. 15 is a graph showing the therapeutic effect of A6-cPS-VCR on in situ MV4-11-GFP-Luc-bearing acute myeloid leukemia mice in vivo bioluminescence fluorescence imaging in Example 11.

FIG. 16 is a graph showing the body weight change and Kaplan-Meier survival curve of each group of micein Example 11.

FIG. 17 shows after dissection the infiltration rates of leukemia cells in the bone marrow, liver and spleen of 3 mice randomly selected from each group.

FIG. 18 is after dissection the HE section diagram of bone marrow, liver and spleen after 3 mice were randomly selected from each group in Example 11.

FIG. 19 is the Micro-CT analysis of the femur after dissection of 3 mice randomly selected from each group in Example 11.

FIG. 20 is a graph showing the therapeutic effect of A6-cPS-DNR on in situ MV4-11-GFP-Luc-bearing acute myeloid leukemia mice by in vivo bioluminescence fluorescence imaging of mice in Example 12.

FIG. 21 shows the proliferation inhibition and apoptosis experiments of A6-cPS-VCR, cPS-VCR and VCR in AML patient leukemia cells in Example 14.

### INVENTION EMBODIMENTS

### Examples of the Invention

The reversible loading a small molecule drug on the vesicle formed by cross-linked biodegradable polymer of the present invention is obtained by self-crosslinking while the amphiphilic triblock polymer is self-assembled; the molecular chain of amphiphilic triblock polymer includes sequentially connected hydrophilic chain segment, hydrophobic chain segment, and PAsp chain segment. The hydrophilic chain segment is PEG with the molecular weight is 3000-8000 Da. The hydrophobic chain segment is PTMC, PLA or PCL with the molecular weight is 2.1-5.7 times that of PEG; the molecular weight of Pasp is 15%-50% that of PEG. The molecular chain of amphiphilic block polymer includes sequentially connected the targeting, hydrophilic chain segment, and hydrophobic chain segment. The molecular weight of PEG is 5000-10000 Da. The hydrophobic chain segment is PTMC, PLA or PCL with the molecular weight is 1.4-3.8 times that of PEG.

### Example 1 Synthesis of Amphiphilic Triblock Polymers:

At First, ring-opening polymerization to synthesize amphiphilic block polymers PEG-P(TMC-DTC), PEG-P(CL-DTC), PEG-P(LA-DTC) ). Then, the terminal hydroxyl groups of the above three amphiphilic block polymers were activated by p-nitrophenyl chloroformate (p-NPC), and then reacted with Pasp to prepare amphiphilic triblock polymers.

Specifically, taking the synthesis of PEG-P(TMC-DTC)-Pasp as an example, the synthetic route is as follows:

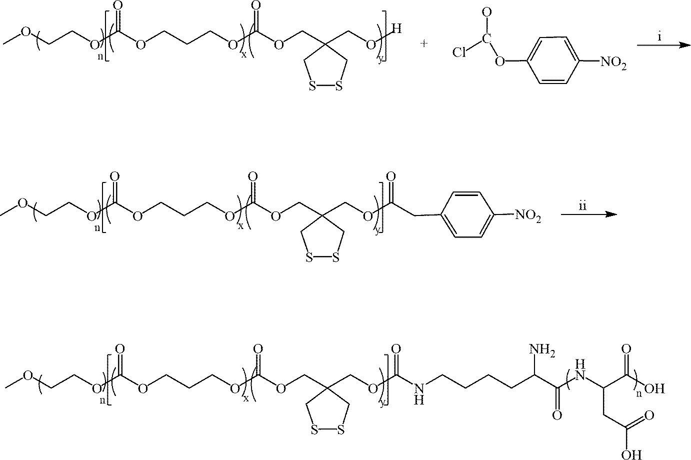

In step (i), the reaction conditions were anhydrous dichloromethane (DCM), pyridine, 25° C., 24 hours; in step (ii), the reaction conditions were anhydrous dimethyl sulfoxide (DMSO), Pasp, triethylamine, 30° C., 48 hours.

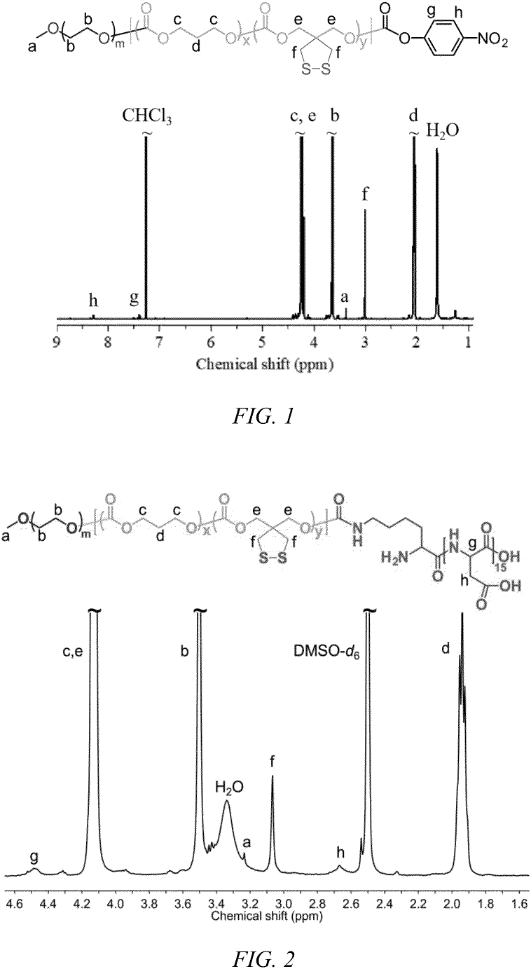

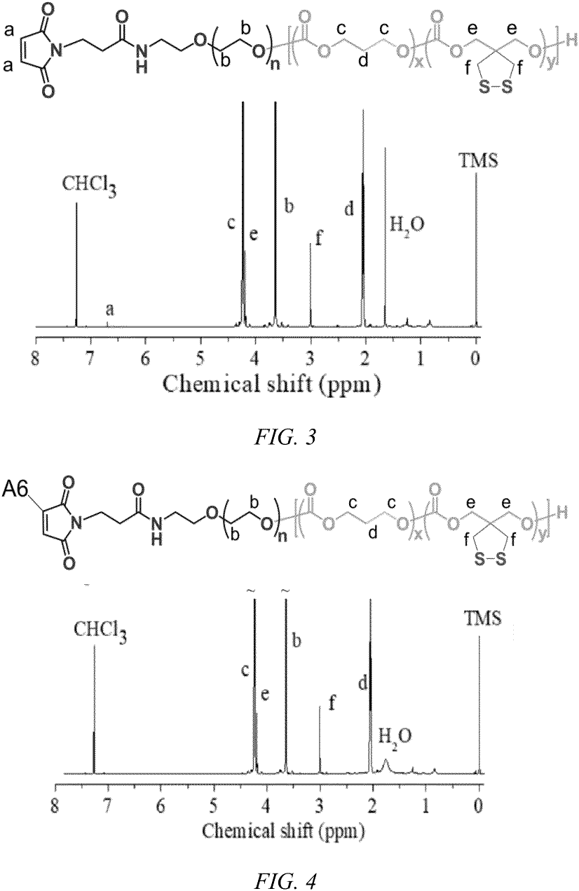

The specific synthesis steps are as follows: The synthesis of PEG-P(TMC-DTC)-Pasp is divided into two steps, namely using p-NPC to activate the end of PEG-P(TMC-DTC) (5.0-(15.0-2.0) kg/mol) After the hydroxyl group, it can be obtained by reacting with Pasp. Taking the synthesis of PEG-P(TMC-DTC)-Pasp (n=15) as an example, the specific operation is as follows. PEG-P(TMC-DTC) (1.0 g, 45.5 µmol) was dissolved in 10 mL of free nitrogen under nitrogen atmosphere. In aqueous DCM, then transferred to an ice-water bath and added pyridine (18.0 mg, 227.5 µmol), and after stirring for 10 minutes, a solution of p-NPC (48.4 mg, 240.3 µmol) in DCM (1.0 mL) was added dropwise. After 30 minutes of dropwise addition, the reaction was continued at room temperature for 24 hours, and then the pyridine salt was removed by suction filtration, and the polymer solution was collected and concentrated to ~100 mg/mL by rotary evaporation, precipitated with glacial ether, and dried in vacuo to obtain the product PEG-P(TMC -DTC)-NPC, yield: 90.0%. Subsequently, under nitrogen protection, Pasp15 (60.0 mg, 83.4 µmol) was weighed and dissolved in 4 mL of anhydrous DMSO and triethylamine (4.2 mg, 41.7 µmol) was added, and then was added dropwise to it with stirring PEG-P (TMC-DTC)-NPC in anhydrous DMSO (9.0 mL) was added dropwise over 30 minutes. After reacting at 30° C. for 2 days, dialyze against DMSO containing 5% anhydrous methanol for 36 hours (replaced the medium 4-5 times) to remove unreacted Pasp and p-nitrophenol produced by the reaction, and then dialyze against DCM for 6 hours. Then the polymer solution was collected and concentrated to a polymer concentration of 50 mg/mL by rotary evaporation, precipitated in ice ether and dried in vacuo to obtain a white cotton-like polymer PEG-P(TMC-DTC)-Pasp, yield: 91.0%. FIGS. 1 and 2 are hydrogen NMR spectra of PEG-P(TMC-DTC)-NPC and PEG-P(TMC-DTC)-Pasp. The characteristic peaks of p-NPC (δ 7.41 and δ 8.30 ppm) and the characteristic peaks of PEG-P(TMC-DTC) (δ 2.03, 2.99, 3.38, 3.63, 4.18 and 4.22 ppm) can be seen from FIG. 1, according to the ratio of the integral area of p-NPC characteristic peak to the area of PEG methyl hydrogen peak at δ 3.38 ppm, the grafting rate of NPC was about 100%. FIG. 3 shows that the characteristic peaks of NPC at δ 7.41 and δ 8.30 ppm were disappeared, and a new signal peak appeared at δ 4.54 ppm, which was the characteristic peak of methine in Pasp. The degree of functionalization of Pasp was calculated to be ~100% by comparing the ratio of the peak area at δ 4.54 ppm to the area of the TMC methylene hydrogen peak at δ 1.95 ppm. The successful synthesis of PEG-P(TMC-DTC)-Pasp was demonstrated for the following examples. PEG-P(TMC-DTC)-Pasp (n = 5, 10) were prepared according to the same method, except that the n value of Pasp was changed.

### Example 2

Synthesis of targeting amphiphilic block polymer: the preparation of targeting amphiphilic block polymer was performed in two steps. The functionalized amphiphilic block polymer with Mal functional group and NHS functional group was synthesized at first, and then the targeted amphiphilic block polymer was obtained by reacting the targeting polypeptide with the functionalized amphiphilic block polymer. Specifically, take A6-PEG-P (TMC-DTC) as an example. Mal-PEG-P(TMC-DTC) (7.5-(14.9-2.1) kg/mol) was firstly synthesized by ring-opening polymerization, and then A6-PEG-P(TMC-DTC) was obtained by the Michael addition reaction of the thiol group of A6 with Mal-PEG-P(TMC-DTC). In the nitrogen, 1 mL of Mal-PEG-P(TMC-DTC) (100 mg, 4.1 µmol) in anhydrous DMSO was added dropwise via a constant pressure dropping funnel to 2 mL of A6 (7.47 mg, 8.2 µmol), and reacted at room temperature for 48 hours. After the reaction, the reaction solution was first dialyzed against DMSO for 36 hours (replacement of the medium 4-5 times) to remove unreacted A6, and then dialyzed against DCM for 6 hours, then the polymer solution was collected and concentrated by rotary evaporation to a polymer concentration of about 50 mg/mL, precipitated in ice ether and dried in vacuo to obtain a white cotton flocculent polymer A6-PEG-P(TMC-DTC). Yield: ~95%. FIG. 3 is the NMR spectrum of Mal-PEG-P (TMC-DTC), according to the integrated area of TMC (δ 2.03, 4.24 ppm) and DTC (δ 3.02, 4.19 ppm) characteristic peaks and PEG characteristic peaks (δ 3.65 ppm) From the ratio of, the degree of polymerization of TMC and DTC was calculated to be 147 and 10, respectively; from the ratio of the characteristic peak of Mal (δ 6.75 ppm) and the integration of PEG methoxy (δ 3.37 ppm), the content of Mal was calculated to be 100%, It shows that Mal remains stable during the reaction and processing. FIG. 4 is the hydrogen NMR spectrum of A6-PEG-P (TMC-DTC), the characteristic peak of Mal at δ 6.75 ppm disappears. In addition, the degree of functionalization of A6 was measured to be approximately 90% using the TNBSA method, indicating the successful synthesis of A6-PEG-P(TMC-DTC), which was used in the following examples.

The synthesis of CLL1 and INGR-targeted amphiphilic block polymers was carried out with reference to the above method, only the Mal-PEG-P(TMC-DTC) polymer was replaced by the NHS-PEG-P(TMC-DTC) polymer. The degree of functionalization is 90~96%.

### Example 3

Preparation of non-targeted drug-loaded polymer vesicles: Non-targeting loading drug on vesicle formed by polymer was prepared by a solvent displacement method, using the electrostatic interaction between the drug and the Pasp chain segment in the amphiphilic triblock polymer. Specifically, the amphiphilic triblock polymer was exemplified by PEG-P(TMC-DTC)-Pasp. PEG-P(TMC-DTC)-Pasp was dissolved in DMSO (40 mg/mL), and 100 µL was dispensed into standing 900 µL HEPES (pH 6.8, 10 mM) containing small molecule drug at 300 rpm After stirring for 3 minutes, incubate at 37° C. for 8 hours. The non- targeting loading drug on vesicle formed by polymer cPS-VCR was obtained by dialysis against HEPES (pH 7.4, 10 mM) for 8 hours.

Among them, the theoretical loading small-molecule drug of VCR was set at 4.8-9.1 wt%, study found that the particle size of the obtained cPS-VCR was about 30 nm, and the particle size distribution was about 0.1 wt% (Table 1). The drug loading of cPS-VCR was calculated to be 4.6-4.9 wt% by measuring its absorbance at 298 nm by UV-Vis spectroscopy. In other methods, the VCR was replaced with cytarabine hydrochloride, and the results showed that the drug loading of cytarabine was 0.1 wt%. The same method was used to load PEG-P(TMC-DTC) vesicles, the results showed that the drug loading of PEG-P(TMC-DTC) vesicle was only 0.7 wt%, that was only about 15% of vesicle cPS-VCR of PEG-P(TMC-DTC)-Pasp; PEG-P(LA-DTC)-Pasp or PEG-P(CL-DTC)-Pasp was used to carry VCR in vesicle by the same method, and the results showed that their drug loading capacity was about 70% of the drug loading of vesicle on PEG-P(TMC-DTC)-Pasp.

TABLE 1

Characterization of cPS-VCR

| Polymer | Particle size (nm)^{a} | PDI^{a} | DLC (wt.%)^{b} | | DLE^{b} (%) |
|---|---|---|---|---|---|
| | | | Theoretical | Test | |
| PEG-P(TMCSTC)-PAsp | 36 | 0.09 | 9.1 | 4.9 | 51.4 |
| | 28 | 0.08 | 7.0 | 4.8 | 66.7 |
| | 28 | 0.11 | 4.8 | 4.6 | 97.2 |
| PEG-P(TMC-DTC) | 75 | 0.12 | 4.8 | 0.7 | 14.1 |

^{a} testing by DLS;
^{b} testing by UV-vis.

The preparation method of the small molecule drug cPS-DNR is the same as the above method. It was found that the particle size of the prepared cPS-DNR was about 28 nm, and the loading drug was 9.4 wt% (when the theoretical drug loading was 16.7 wt%) (Table 2). In addition, on the basis of the above loading drug method, the loading drug effect was studied by changing the pH value and incubation time of HEPES, and the results showed that the drug loading amount did not change significantly. Compared with PEG-P(TMC-DTC), PEG-P(LA-DTC) or PEG-P(CL-DTC), the loading vesicle with DNR by the same method, and the results showed that the drug loading of this vesicle was only less than 50% that of PEG-P(TMC-DTC)-Pasp vesicles.

The preparation method of the small molecule drug mitoxantrone polymer vesicle Is the same as the above method. The results showed that the prepared mitoxantrone-loaded polymersomes (cPS-MTO) had a particle size of 50-130 nm due to different dosages. The drug loading of cPS-MTO was 3.3-9.1 wt% (Table 3). PEG-P(TMC-DTC) vesicles were loaded with MTO by the same method, and the results showed that the drug loading of PEG-P(TMC-DTC) vesicles was as low as 0.3-0.7 wt%.

To sum up, it is concluded that the amphiphilic triblock polymer of the present invention has unexpected technical effects for loading small molecule drugs.

TABLE2

Characterization of cPS-DNR

| Polymer | Particle size (nm)^{a} | PDI^{a} | DLC (wt.%)^{b} | | DLE^{b} (%) |
|---|---|---|---|---|---|
| | | | Theoretical | Test | |
| PEG-P(TMC-DTC)-PAsp | 28 | 0.19 | 16.7 | 9.4 | 52.1 |
| | 27 | 0.14 | 9.1 | 5.5 | 52.6 |
| PEG-P(TMC-DTC) | 54 | 0.18 | 16.7 | 4.8 | 25.4 |
| | 52 | 0.15 | 9.1 | 2.2 | 24.2 |

^{a} testing by DLS;
^{b} testing by UV-vis.

TABLE3

characterization of cPS-MTO

| Polymer | Particle (nm)^{a} | PDI^{a} | DLC (wt.%)^{b} | | DLE^{b} (%) |
|---|---|---|---|---|---|
| | | | Theoretical | Test | |
| PEG-P(TMC-DTC)-PAsp | 58 | 0.11 | 5.0 | 3.3 | 69 |
| | 81 | 0.17 | 10.0 | 6.4 | 68 |
| | 126 | 0.28 | 15.0 | 9.1 | 67 |
| PEG-P(TMC-DTC) | 59 | 0.11 | 5.0 | 0.3 | 2.9 |
| | 62 | 0.12 | 10.0 | 0.3 | 3.1 |
| | 65 | 0.10 | 15.0 | 0.7 | 3.5 |

^{a} testing by DLS;
^{b} testing by UV-vis.

### Example 4

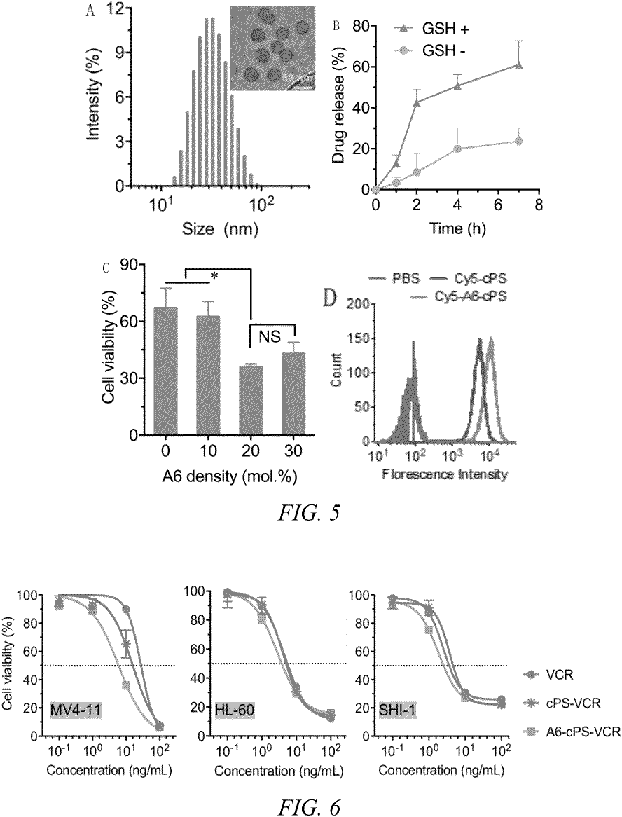

Preparation of targeting loading a drug on the vesicle formed by polymer: The targeting loading a drug on the vesicle formed by polymer was composed of the amphiphilic triblock polymer synthesized in Example 1 and the targeting amphiphilic polymer in Example 2, drug co-assembly prepared by solvent replacement method. Specifically, take the VCR-loaded A6 polypeptide-directed polymersome (A6-cPS-VCR) as an example. The DMSO solution of A6-PEG-P(TMC-DTC) and PEG-P(TMC-DTC)-Pasp polymers (total polymer concentration was 40 mg/mL, in which A6-PEG-P(TMC-DTC) and the molar ratio of PEG-P(TMC-DTC)-Pasp to obtain different ratios of A6-cPS-VCR), put 0.5 mL into 4.5 mL HEPES (10 mM, pH 6.8) buffer containing VCR, and stirred at 300 rpm for 5 min. A6-cPS-VCR with different ratios was obtained by dialysis with HEPES (pH 7.4, 10 mM) for 8 hours after incubation at 37° C. In the targeting loading a drug on the vesicle formed by polymer, the content of A6-PEG-P(TMC-DTC) polymer was 10.0-30.0 mol.%. The results showed that the particle size of A6-cPS-VCR was 36-47 nm (FIG. 5A), the particle size distribution was narrow (0.05-0.11), and the drug loading efficiency of VCR was between 79.8%-84.3% (Table 4).

TABLE 4

characterization of A6-cPS-VCR

| ratio of A6-PEG-P(TMC-DTC) | Particle^{a} size (nm) | PDI^{a} | DLC (wt.%) ^{b} | | DLE^{b} (%) |
|---|---|---|---|---|---|
| | | | Theoretical | Test | |
| 10% | 42 | 0.05 | 4.8 | 4.0 | 84.3 |
| | 45 | 0.07 | 4.8 | 3.8 | 80.8 |
| 30% | 47 | 0.09 | 4.8 | 3.8 | 79.8 |

^{a} testing by DLS;
^{b} testing by UV-vis.

The same method can be used to prepare the carrier based on 20% of A6-PEG-P(TMC-DTC) polymer and 80% of PEG-P(TMC-DTC)-Pasp5 or PEG-P(TMC-DTC)-Pasp10, respectively. VCR vesicle, when the theoretical drug loading amount was 4.8 wt%, obtained VCR with loading drug efficiencies was 62.4% and 73.3%, and particle sizes was 58 nm and 52 nm, respectively.

### Example 5

In vitro drug release of A6-cPS-VCR-targeted polymer vesicle nanomedicine: 20% A6-cPS-VCR was used as a representative to study the in vitro drug release behavior of A6-cPS-VCR-targeted vesicle nanomedicine. The in vitro drug release behavior of A6-cPS-VCR was studied by dialysis with two release media, HEPES (pH 7.4, 10 mM) and HEPES solution containing 10 mM GSH (nitrogen atmosphere). 0.5 mL of A6-cPS-VCR (0.5 mg/mL) was first loaded into a release bag (MWCO: 14 kDa) and then placed in 20 mL of the corresponding release medium in a shaker at 37° C., 100 rpm. At the set time points (0, 1, 2, 4, 7 h), 5 mL of dialysate was withdrawn and supplemented with 5 mL of fresh medium. The content of VCR in the dialysate was determined by HPLC (mobile phase was methanol: water (15% triethylamine was added, pH was adjusted to 7.0 with phosphoric acid) = 70:30). FIG. 5B is a graph showing the results of In vitro release of A6-cPS-VCR targeting vesicle nano-drug. The results showed that under 10 mM of GSH, A6-cPS-VCR released more than 60% of VCR within 7 hours, while under non-reducing conditions, the cumulative release of VCR within 24 hours was only about 23%.

### Example 6

A6-cPS-VCR-targeted polymer vesicle nanomedicine inhibits the proliferation of MV4-11 cells: Firstly, prepared A6-cPS-VCR with different surface densities of A6, namely cPS-VCR (refer to preparation method of Example 3 )and 10% of A6-cPS-VCR, 20% of A6-cPS-VCR, 30% of A6-cPS-VCR (refer to preparation method of Example 4 ), and by the cell proliferation inhibition assay (CCK8 method) to study the effects of acute myeloid cells with high CD44 expression. Firstly, MV4-11 cells were plated in a 96-well plate (2×10⁴ cells/well), placed in an incubator for 24 hours, and then 20 µL of 10% A6-cPS-VCR and 20% of A6-cPS-VCR were added to the wells. 30% of A6-cPS-VCR or cPS-VCR (10 ng/mL in VCR well), and the control group were supplemented with 20 µL of PBS. After 4 hours of incubation, the supernatant was removed by centrifugation (3000 rpm, 10 minutes), then fresh RPMI-1640 complete medium was added, the cells were blown off and placed in the incubator for a further 44 hours. 10 µL of CCK8 was added and the incubation continued for 2 hours. Finally, the absorbance at 450 nm wavelength was detected by a microplate reader. The cell viability was calculated by the ratio of the absorbance value of the experimental group to the absorbance value of the control group, and the experiment was performed in parallel with three replicate wells (mean ± SD, n = 3). The test results showed that 20% of A6-cPS-VCR had the highest proliferation inhibitory effect (FIG. 5C). In the following implementation, unless otherwise specified, A6-cPS-VCR was 20% that of A6-cPS-VCR.

### Example 7

Endocytosis behavior of A6-cPS-VCR-targeted polymer vesicle nanomedicine: using Cy5-labeled polymer (refer to Example 2 for preparation method, replace A6 with Cy5), and A6-PEG-P (TMC -DTC) and PEG-P(TMC-DTC)-Pasp were mixed at 0.5:20:79.5 to prepare Cy5-A6-cPS and Cy5-cPS (refer to Example 4 for the preparation method), and A6-cPS was studied by flow cytometry. By cPS-VCR in acute myeloid leukemia cell line MV4-11 with high CD44 expression. Firstly, MV4-11 cells were plated in a 6-well plate (2×10⁵ cells/well), placed in an incubator for 24 hours, and then 200 µL of Cy5-A6-cPS or Cy5-cPS (concentration of Cy5 was 2.0 µg/mL) was added, the control group was added with 200 µL PBS. After 4 hours of incubation, cells were collected by centrifugation (800 rpm, 5 min), washed twice with PBS, and finally dispersed with 500 µL of PBS and placed in a flow tube for assay. The test results showed (FIG. 5D) that the endocytosis of Cy5-A6-cPS in MV4-11 cells was significantly higher than that of Cy5-cPS, and its fluorescence intensity was twice that of the Cy5-cPS group, indicating that the introduction of A6 polypeptide can significantly increase the amount of Cy5-A6-cPS. Enhanced cellular uptake of Cy5-cPS.

### Example 8

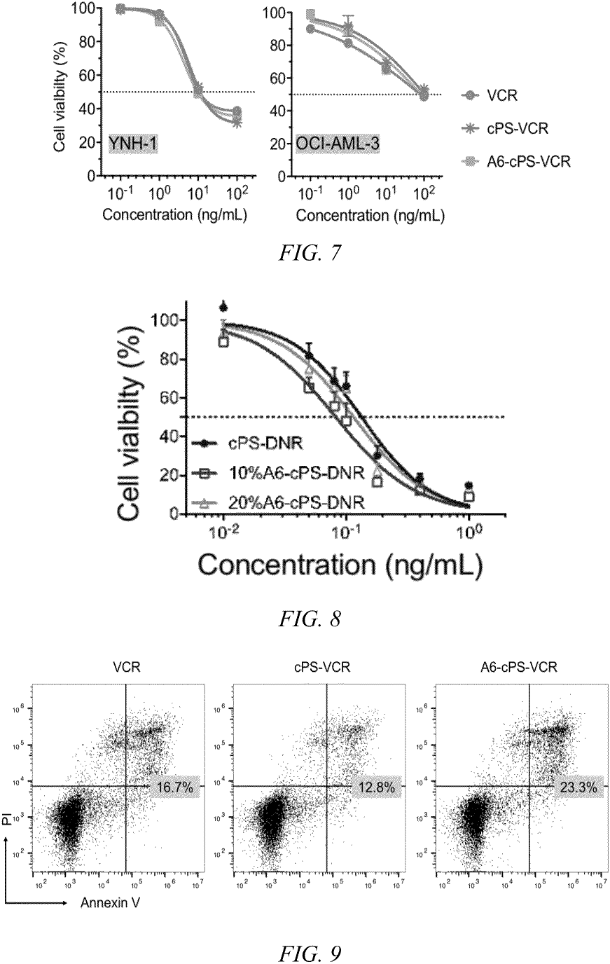

Inhibition of cell proliferation by A6-cPS-VCR-targeted polymer vesicle nanomedicine: Inhibition of cell proliferation by A6-cPS-VCR on CD44-positive acute myeloid leukemia cell lines MV4-11, HL-60 and SHI-1 The CCK8 method was used for determination. The cells were first plated in 96-well plates (2×10⁴ cells/well) and cultured for 24 hours, then 20 µL of A6-cPS-VCR, cPS-VCR or free VCR were added (the final concentrations of VCR were 0.001, 0.01, 0.1, and 1, respectively), 10 and 100 ng/mL), and the control group was supplemented with 20 µL of PBS. After 4 hours of incubation, the supernatant was removed by centrifugation (3000 rpm, 10 minutes), then fresh RPMI-1640 complete medium was added, the cells were blown off and placed in the incubator for a further 44 hours. Add 10 µL of CCK8 and continue to incubate for 2 hours. Finally, the microplate reader detects the absorbance at 450 nm wavelength. Cell viability = (absorbance value of experimental group - absorbance of blank)/(absorbance value of control group - absorbance of blank) × 100%, and the experiment was carried out in parallel with three replicate wells (mean ± SD, n = 3). FIG. 6 shows the results of inhibition of proliferation of MV4-11, HL-60 and SHI-1 cells by A6-cPS-VCR, cPS-VCR and VCR. The results showed that the median inhibitory concentration (IC₅₀) of A6-cPS-VCR was lower than that of cPS-VCR and VCR in all three cell lines. A6-cPS-VCR, cPS-VCR and VCR had no significant difference in the proliferation inhibition of CD44-negative cell lines YNH-1 and OCI-AML-3 (FIG. 7).

The cell proliferation inhibition of MV4-11 by A6-cPS-DNR refers to the above method. Only added drugs were replaced with A6-cPS-DNR and cPS-DNR. FIG. 8 shows the results of inhibition of proliferation of MV4-11 by A6-cPS-DNR and cPS-VCR. The results showed that 10% A6-cPS-DNR had the lowest IC₅₀.

### Example 9

A6-cPS-VCR-targeted polymer vesicle nanomedicine induces apoptosis and cell cycle: First, MV4-11 cells were plated in a 24-well plate at a density of 2 × 10⁵ cells/well and cultured for 24 hours. 20 µL of A6-cPS-VCR, cPS-VCR or VCR (concentration of VCR was 10 ng/mL) was added, respectively, and 20 µL of PBS was added to the control group. After 4 hours of incubation, the supernatant was removed by centrifugation (3000 rpm, 10 minutes), then fresh RPMI-1640 complete medium was added, the cells were blown off and placed in the incubator for a further 44 hours. Cells were collected in a flow tube, centrifuged (800 rpm, 5 min) and washed twice with 4° C. cold PBS. Finally, 200 µL of binding buffer was added to resuspend the cells. After pipetting evenly, taken 100 µL of it into the flow tube, added 5 µL of AnnexinV-F647 and 10 µL of PI solution in sequence, and stain at room temperature for 15 minutes in the dark, then add 400 µL of PBS and mix evenly to stop the staining, and detection by a flow cytometer within 1 hour. Among them, the cells that were treated in a 50° C. water bath for 5 minutes and fixed with 4% paraformaldehyde for 5 minutes were added with 5 µL of AnnexinV-F647 solution and 10 µL of PI solution for staining for 15 minutes respectively. Cytometer for testing. FIG. 9 shows the results of A6-cPS-VCR-induced apoptosis of MV4-11 cell line. The results showed that A6-cPS-VCR could effectively induce apoptosis. When the concentration of VCR was 10 ng/mL, it could induce 23.3% apoptosis, and the apoptosis rate was significantly higher than that in the non-targeted control cPS-VCR group (12.8%) and free VCR group (16.7%).

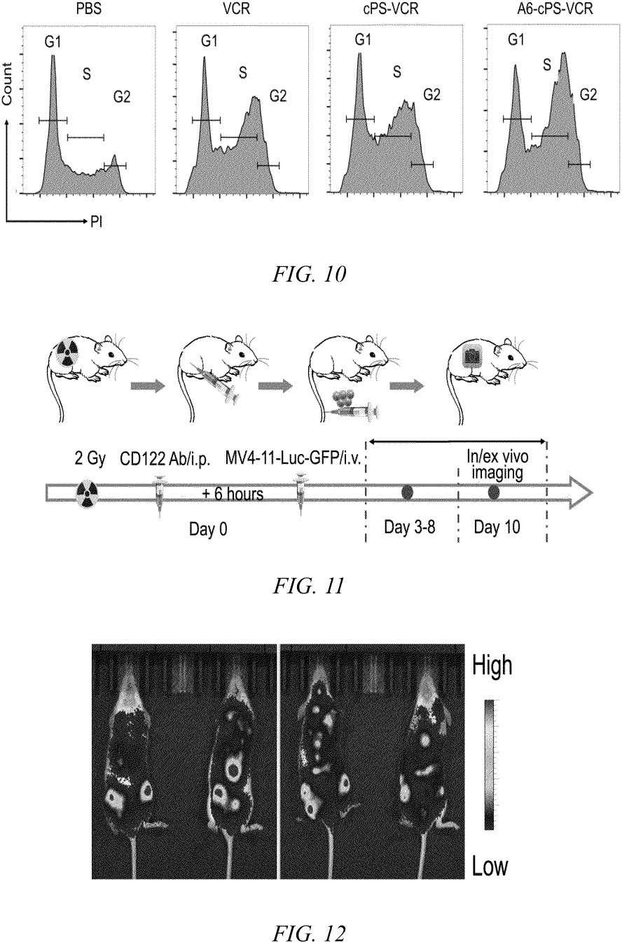

The cell culture and treatment of the cell cycle experiment of A6-cPS-VCR refer to the above method, and only add 1 mL of PBS to resuspend the cells at the end. Then, 4 mL of ice 95% ethanol was added to fix the cells for 12 hours, and 400 µL of PI staining solution was added for 30 minutes in the dark on a shaker at 37° C. Finally, the cells were tested by flow cytometry. FIG. 10 shows the cycle results of A6-cPS-VCR in MV4-11 cell line. The results showed that A6-cPS-VCR and VCR have similar mechanisms, arresting cells in G2/M phase, and eventually leading to apoptosis.

### Example 10

Construction of an in situ MV4-11-GFP-Luc acute myeloid leukemia mouse model: All animal experiments and operations were approved by the Experimental Animal Center of Soochow University and the Animal Care and Use Committee of Soochow University. Establishment of an orthotopic acute myeloid leukemia model: As shown in FIG. 11, the establishment of an orthotopic acute myeloid leukemia model requires the use of NOD/SCID strain mice (6 weeks old, female, body weight greater than 20 g). Mice were first irradiated with X-rays (2.0 Gy), then the antibody CD122 (10 mg/kg mouse) was injected intraperitoneally, and 6 hours later, MV4-11-GFP-Luc cells (1×10⁵ cells/mouse) injected into mice by tail vein. After inoculation, the proliferation and proliferation of leukemia cells in mice were observed by in vivo imaging of small animals (FIG. 12).

### Example 11

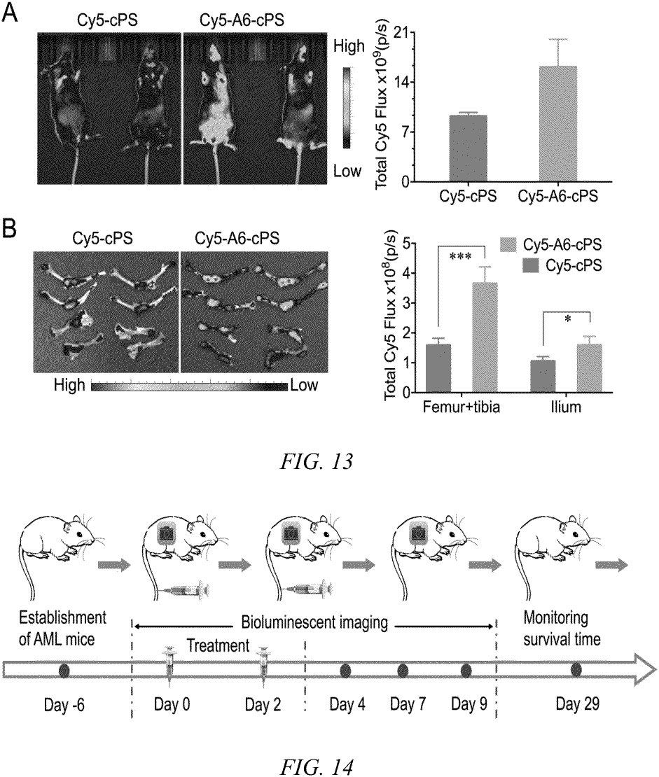

Enrichment of Cy5-A6-cPS in orthotopic MV4-11-GFP-Luc mice: Targeted enrichment of Cy5-A6-cPS in the bone marrow of orthotopic MV4-11-GFP-Luc mice The collection situation was obtained by in vivo and ex vivo Cy5 fluorescence imaging analysis. On the 10^{th} day after inoculation, 200 µL of Cy5-A6-cPS and Cy5-cPS (250 µg Cy5equiv./kg) were injected into the mice through the tail vein, respectively, and the mice were anesthetized using an isoflurane gas anesthesia system for 8 hours. In vivo Cy5 fluorescence imaging (FIG. 13A). The mice were then dissected to remove their femurs, tibias and ilium for ex vivo fluorescence imaging (FIG. 13B). The results showed that Cy5-A6-cPS could be efficiently targeted and enriched in the bone marrow, and the fluorescence signal was significantly higher than that of the non-targeted Cy5-cPS group.

### Example 12

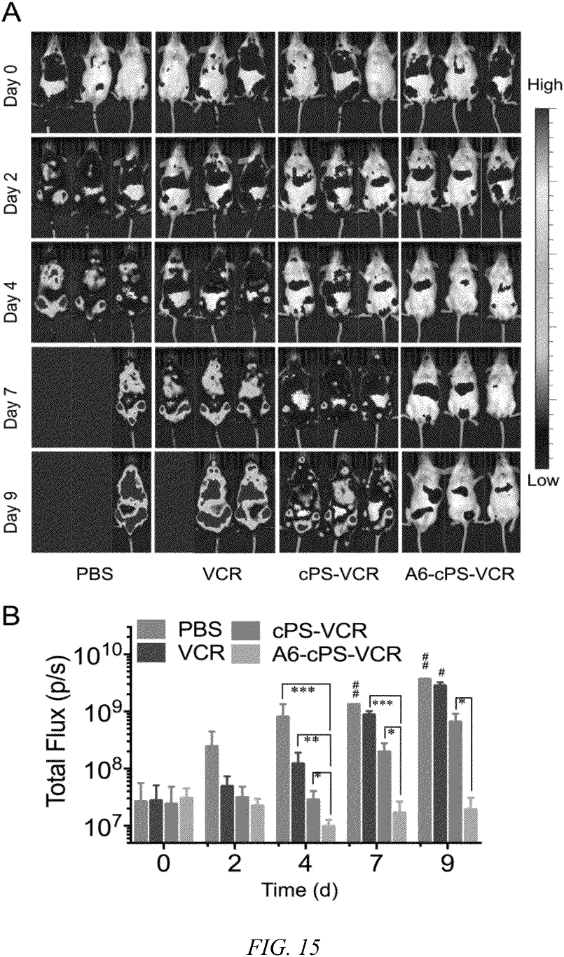

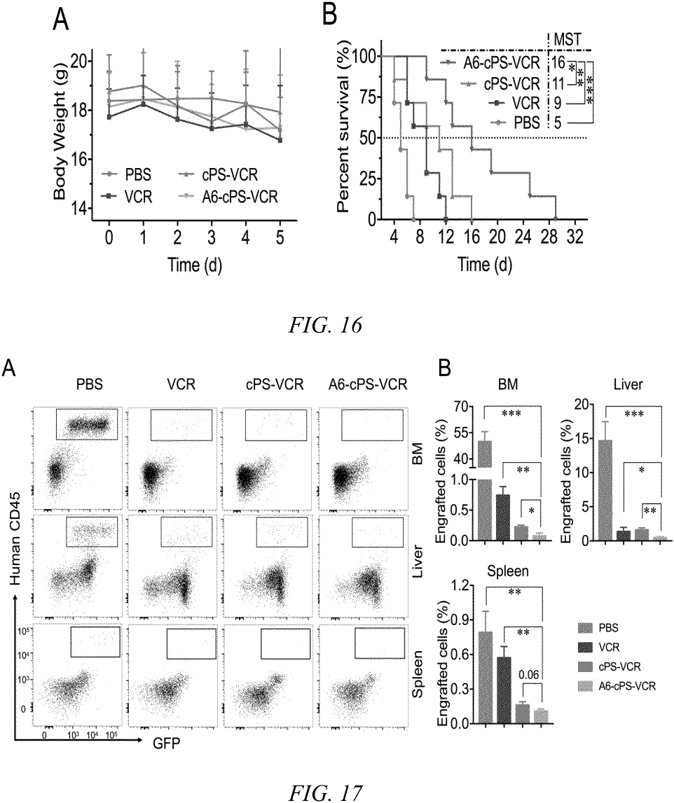

Antitumor effect of A6-cPS-VCR in orthotopic MV4-11-GFP-Luc-bearing mice: To study the antitumor effect of A6-cPS-VCR on orthotopic MV4-11-GFP-Luc bearing mice Tumor effect, the following treatment plan was designed (FIG. 14): the first injection was injected on the 6^{th} day after the inoculation, which was counted as the 0^{th} day, and the second injection was given on the 2^{nd} day. The dose of VCR was 0.50 mg/kg, and the control group was injected with 100 µL PBS. There were 10 tumor-bearing mice in each group. Typical images of mouse bioluminescence fluorescence imaging (FIG. 15A) and quantitative results (FIG. 15B) showed that the MV4-11-GFP-Luc cells in the blank control group continued to proliferate rapidly, and began to die on the 4^{th} day. The MV4-11-GFP-Luc cells of the mice in the VCR group proliferated slowly during drug administration, but proliferated rapidly after drug withdrawal. The MV4-11-GFP-Luc cells of the mice in the cPS-VCR group stopped proliferating during drug administration, but resumed proliferation after drug withdrawal. The MV4-11-GFP-Luc cells of the mice in the A6-cPS-VCR group were killed during the administration period and proliferated slightly after drug withdrawal, indicating that A6-cPS-VCR could effectively inhibit abnormal proliferation of MV4-11-GFP-Luc in mice. There was no significant decrease In body weight In each group of mice during the administration period (FIG. 16A), indicating that the mice tolerated this dose well. Mice lose weight before dying. In addition, the survival time of the mice in the A6-cPS-VCR group was significantly prolonged (FIG. 16B), and the median survival time of the mice was 16 days, compared with the PBS group (6 days) and the VCR group (9 days). And cPS-VCR (11 days), prolonged 1.5-2.7 times.

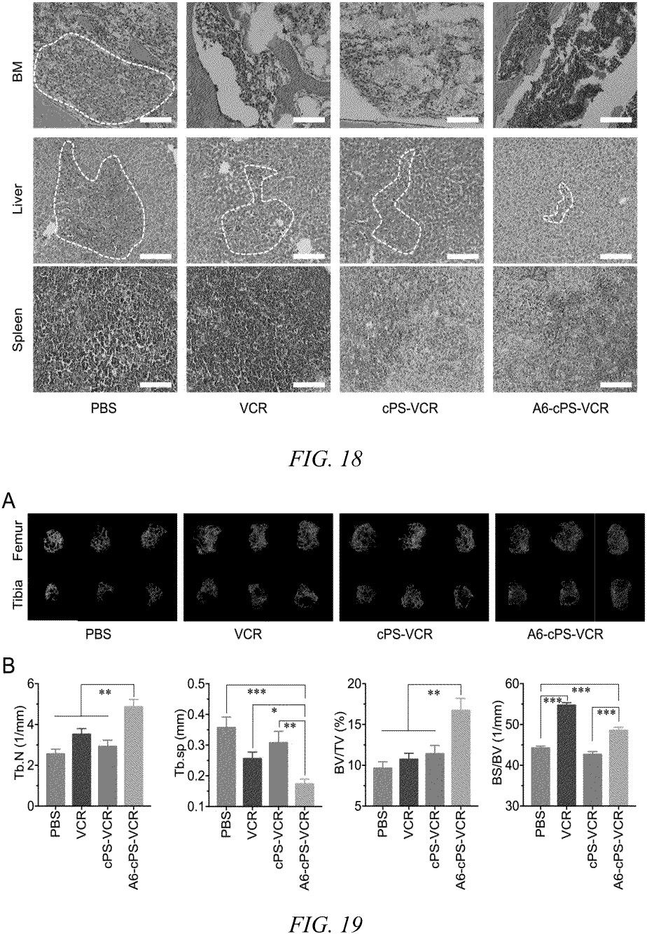

In order to further accurately and quantitatively analyze the proliferation of leukemia cells in mice, on the 4^{th} day, 3 mice in each group were randomly selected for dissection, and the leukocytes in the liver, spleen and bone marrow were extracted, and the content of leukemia cells was detected by flow cytometry. And the liver, spleen and bone marrow slices were analyzed to observe the damage. As shown in FIG. 17, up to about 55% of leukemia cells were detected in the bone marrow of mice in the PBS group, and different degrees of leukemia cells were also detected in the liver and spleen, indicating that the model had a high degree of malignancy. The leukemia levels in the liver, spleen and bone marrow of mice in the VCR and cPS-VCR groups were reduced, but compared with the A6-cPS-VCR group, the A6-cPS-VCR showed better antitumor effect . The HE staining sections (FIG. 18) showed that the bone marrow and spleen tissues of the A6-cPS-VCR group were almost normal, and the liver tissues were almost normal, which also indicated that A6-cPS-VCR had better anti-tumor effect.

Acute myeloid leukemia cells abnormally proliferate in the bone marrow of mice, resulting in osteolytic lesions in mice. Therefore, micro-CT was used to evaluate the relevant indexes of the femur of each group of mice. The results (FIG. 19) found that there were severe osteoclasts in the hind leg bones of the mice in the PBS group and the cPS-VCR group, and a large number of bone trabeculae were missing. After A6-cPS-VCR treatment, the osteolytic lesions of the mice were significant improvement improved.

### Example 13

Antitumor effect of A6-cPS-DNR in orthotopic MV4-11-GFP-Luc-bearing mice: To study the antitumor effect of A6-cPS-DNR on orthotopic MV4-11-GFP-Luc-bearing mice Tumor effect (refer to Example 11 for the design scheme). The DNR dose was 2 mg/kg, and A6-cPS-DNR was increased by 3 mg/kg in a high-dose group. The blank control group was injected with 100 µL PBS. There were 3 tumor-bearing mice in each group.

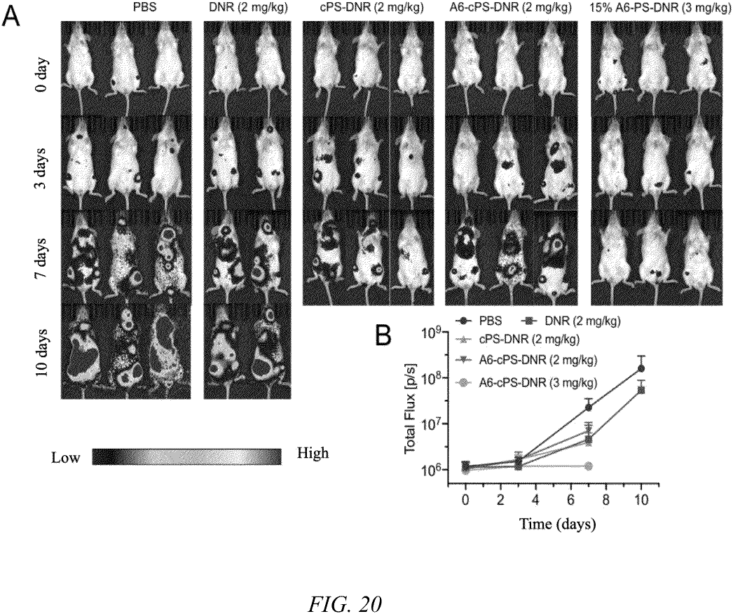

The typical Images of mouse bioluminescence fluorescence Imaging (FIG. 20A) and quantitative results (FIG. 20B) showed that the MV4-11-GFP-Luc cells in the blank control group continued to proliferate rapidly, and the fluorescence quantification in the DNR group decreased slightly, but Treatment is ineffective. The fluorescence quantification of mice in the A6-cPS-DNR and cPS-DNR groups at 2 mg/kg was similar to that in the DNR group. The A6-cPS-DNR high-dose group had obvious therapeutic effect, but the mice in this group died quickly. This experiment shows that A6-cPS-DNR is highly toxic and has a narrow therapeutic window, and the use of A6-targeted polymer vesicles for the treatment of AML is not suitable for all drugs, and specific problems need to be analyzed.

### Example 14

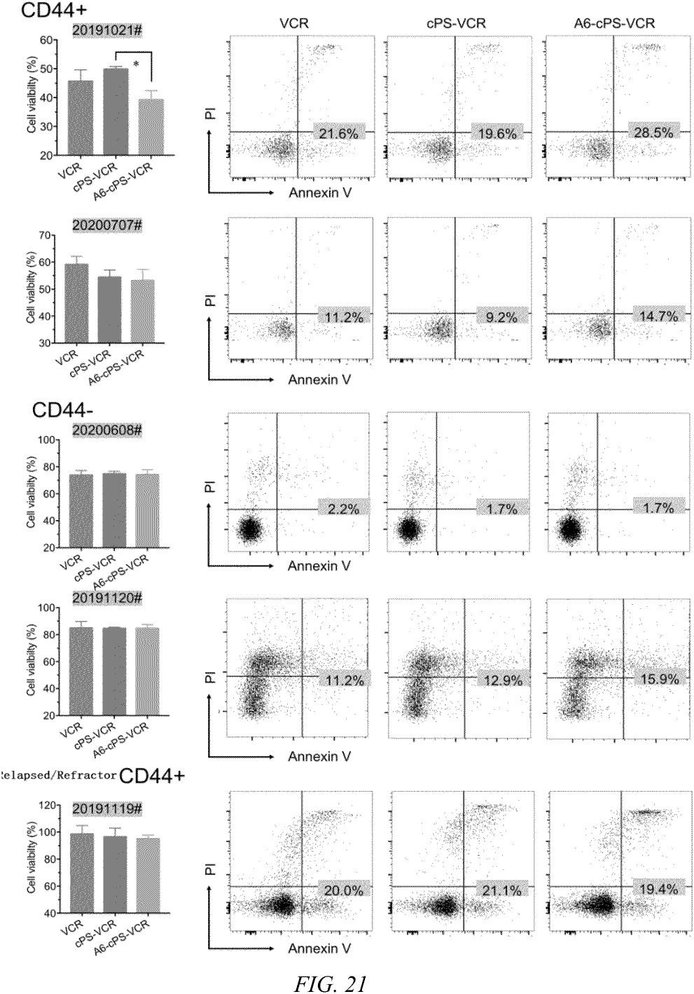

Inhibitory effect of A6-cPS-DNR on proliferation and apoptosis of primary cells of AML patients: Inhibition of proliferation of primary acute myeloid leukemia cells derived from clinical AML patients by A6-cPS-VCR using trypan blue measured by counting method. Firstly, the patient primary cells were plated in a 96-well plate (2×10⁴ cells/well), and 20 µL of A6-cPS-VCR, cPS-VCR or free VCR (the final concentration of VCR was 1 mg/mL, respectively) was added to the control. Add 20 µL of PBS to the group. After 4 hours of incubation, the supernatant was removed by centrifugation (3000 rpm, 10 minutes), then fresh RPMI-1640 complete medium was added, the cells were blown off and placed in the incubator for a further 44 hours. The number of viable cells in each well was counted with trypan blue dye. Cell survival rate = (the number of viable cells in the experimental group - the number of blank viable cells)/ (the number of viable cells in the control group - the number of blank viable cells) × 100%. The results (FIG. 21) showed that the toxicity of A6-cPS-VCR to CD44+ patient primary cells was higher than that of CD44- primary cells, and its toxicity to CD44+ cells was higher than that of cPS-VCR, showing a certain targeted therapeutic effect, while There was no significant targeting effect on CD44-patient cells.

The apoptosis experiment method of A6-cPS-VCR on primary AML cells of patients refers to the apoptosis experiment of MV4-11 cell line in Example 9. Only the concentration of A6-cPS-VCR, cPS-VCR or VCR of VCR was replaced by 1 mg/mL). The results (FIG. 21) show that A6-cPS-VCR has a certain targeted therapeutic effect on primary cells of CD44+ patients, but has no obvious targeting effect on CD44- patients. Apoptosis of patient cells was consistent with the results of proliferation inhibition experiments.

In conclusion, the reversible cross-linking reduction-sensitive drug-loaded polymer vesicle nano-drug with asymmetric membrane structure of the present invention can efficiently load vincristine sulfate in the cavity, can significantly improve the curative effect of acute myeloid leukemia in situ, and at the same time its biological It has the advantages of degradation, in vivo safety, and simple preparation process, and has the prospect of clinical transformation.

1. A peptide targeting drug-load polymer vesicle having an asymmetric structure, which is obtained by co-loading an amphiphilic triblock polymer and a peptide targeting amphiphilic block polymer with a small molecule drug; or obtained by co-loading the amphiphilic triblock polymer and a functionalized amphiphilic block polymer with the small molecule drugs and then followed by a peptide preparation;
wherein the small molecule drug includes vincristine sulfate, daunorubicin or mitoxantrone; the chemical structural formula of the amphiphilic triblock polymer is as follows: 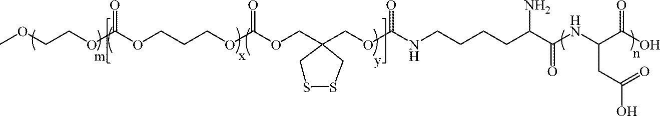 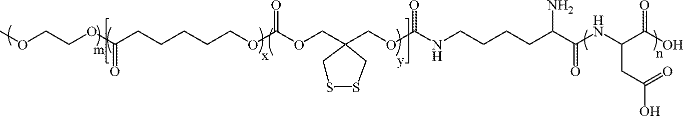 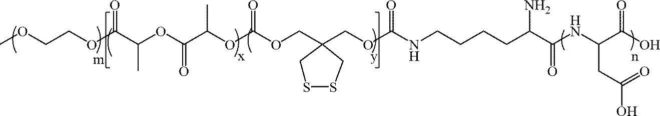
wherein, n is 5-20.

2. The peptide targeting drug-load polymer vesicle having an asymmetric structure according to claim 1, wherein: in the amphiphilic triblock polymer, a molecular weight of a hydrophilic chain segment is 3000-8000 Da; a molecular weight of the hydrophobic chain segment is 2.5-6 times that of the a chain segment; a molecular weight of a PDTC chain segment is 8%-30% of that of the hydrophobic chain segment.

3. Use of the peptide targeting drug-load polymer vesicle having an asymmetric structure according to claim 1 in preparing an anti-tumor drug.

4. The use according to claim 3, wherein the peptide is A6, CLL1 or iNGR.

5. Use of a drug-load polymer vesicle having an asymmetric structure in preparing an anti-acute myeloid leukemia drug; the drug-load polymer vesicle having an asymmetric structure is obtained by loading an amphiphilic triblock polymer and an amphiphilic block polymer with a small molecular drug; the small molecule drug includes vincristine sulfate, daunorubicin or mitoxantrone;
the chemical structural formula of the amphiphilic triblock polymer is as follows: 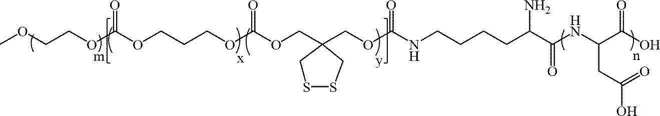 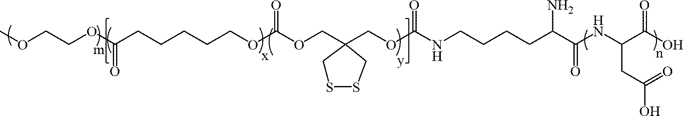 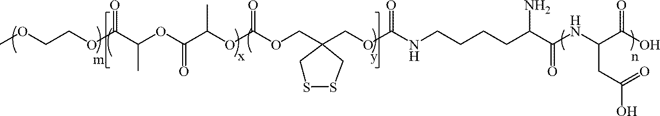
wherein, n is 5-20.

6. The use according to claim 5, wherein the method for preparing the drug-load polymer vesicle having an asymmetric structure comprises the following steps: preparing the non-targeting loading drug on the vesicle formed by polymer by a solvent displacement method using the small molecule drug and the amphiphilic triblock polymer as starting materials.

7. A method for preparing the peptide targeting drug-load polymer vesicle having an asymmetric structure according to claim 1, wherein the method comprises the following steps:
preparing the targeting loading drug on the vesicle formed by polymer by a solvent displacement method using the small molecule drug, the amphiphilic triblock polymer and targeting amphiphilic block polymer as starting materials;
or preparing the loading drug on the vesicle formed by polymer by a solvent displacement method using the small molecule drug, the amphiphilic triblock polymer and the functionalized amphiphilic block polymer as raw materials, then preparing the targeting loading drug on the vesicle formed by polymer by peptide reaction.

8. The method according to claim 7, wherein, the an of the targeted amphiphilic block polymer is 5%-35% of that of the molar sum of the amphiphilic triblock polymer and targeting amphiphilic block polymer; the amount of the functionalized amphiphilic block polymer is 5%-35% of that of the molar sum of the amphiphilic triblock polymer and targeting amphiphilic block polymer.

9. The method according to claim 7, wherein the peptide is A6, CLL1 or iNGR.

10. Use of the peptide targeting drug-load polymer vesicle having an asymmetric structure according to claim 1, the active ingredients of the drug are vincristine sulfate, daunorubicin or mitoxantrone.
